(19) United States
(12) Patent Application Publication
(10) Pub. No.: US 2011/0021883 A1
(43) Pub. Date: Jan. 27, 2011

(54) Remote health care system for enabling user to select a symptom diagnosis algorithm appropriate to his/her individual disease and method thereof

(75) Inventors: Shuenn-Tsong Young, Hsinchu County (TW); Chung-Wang Lee, Hsinchu County (TW); Chang-Yu Liu, Hsinchu County (TW)

Correspondence Address: BACON & THOMAS, PLLC, 625 SLATERS LANE, FOURTH FLOOR, ALEXANDRIA, VA, 22314-1176, US

(73) Assignee: Aescu Technology Inc., Hsinchu County (TW)

(21) Appl. No.: 12/654,655

(22) Filed: Dec. 29, 2009

(30) Foreign Application Priority Data
Jul. 21, 2009 (TW) 098124530

(51) Int. Cl. A61B 5/00; G06Q 50/00; G06Q 30/00

(52) U.S. Cl. 600/300; 705/2

(57) ABSTRACT

This invention relates to a remote health care system and a method thereof for allowing medical experts specializing in various medical fields to upload various developed program files of symptom diagnosis algorithms to a database of a care server in the remote health care system for adding, revising or updating the program files stored therein, so that the care server can provide a variety of new and useful program files to a user and allow the user to select the program files more appropriate to his/her individual disease and to upload his/her physiological parameter data to the care server. Then, the care server can execute corresponding calculating and analyzing procedures to the uploaded physiological parameter data by using the symptom diagnosis algorithm in the selected program file, and transmit calculation and analysis results to the user or health care workers for precisely and objectively understand the user's individual physiological condition.

### FIELD OF THE INVENTION

The present invention relates to a remote health care system for enabling a user to select a symptom diagnosis algorithm appropriate to his/her individual disease, more particularly to a remote health care system allowing medical experts specializing in various medical fields to upload various developed program files of symptom diagnosis algorithms to a database of a care server in the remote health care system, so that user can select the program file more appropriate to his/her individual disease from the care server and upload physiological parameter data of the user to the care server for calculating and analyzing the physiological parameter data by using the selected program file and enabling the user or health care workers to immediately obtain corresponding calculation and analysis results and to precisely and objectively understand the user's individual physiological condition.

### BACKGROUND OF THE INVENTION

Recently, with the improvement of human life qualities and economic conditions, people not only seek comfortable life environments or physical lives, but also pay more and more attention to their own physiological health conditions with the hope that they can healthily live and have longevities. Thus, related manufacturers of medical health-care foods, equipments or appliances focus on this business opportunity, and invest great efforts and funds to develop and research various medical health-care foods, equipments or appliances, in order to significantly improve users' bodies and symptoms and attract attentions of consumers for earning relative profits from these medical health-care foods, equipments or appliances.

For example, a traditional remote health care system is developed under this trend. The remote health care system comprises a care server and at least one physiological parameter detection device, wherein the physiological parameter detection device can be carried by a user or installed on a specific location (such as installed beside a sickbed). The physiological parameter detection device can continuously detect and capture physiological parameter data of the user (such as electrocardiograms, blood pressure data, blood glucose data, blood oxygen data, etc.), and link to the care server via wired networks (such as ADSL, optical fibers, etc.) or wireless networks (such as 802.11, WiMAX, WiFi, 3G, etc.) for transmitting the detected and captured physiological parameter data to the care server. Then, the care server can store the physiological parameter data in one of databases therein, and analyze the physiological parameter data by a preset symptom diagnosis algorithm stored in the care server (such as an arrhythmia algorithm for calculating and analyzing whether or not the heartbeat frequency is stable within a predetermined duration, a heartbeat algorithm for calculating and analyzing the heartbeat number per minute, etc.). Finally, the analyzed result is transmitted to the physiological parameter detection device, so that the user can know his/her own physiological condition for implementing remedial measures (such as sports, food therapies, drug administration, etc.). Alternatively, the care server also can send the analyzed result back to another database of the care server, so that health care workers can immediately determine the user's physiological condition according to the physiological parameter data and the analyzed data stored in the care server. More importantly, when the user is detected to be in a physical uncomfortableness or danger, the health care workers can immediately implement an appropriate therapy and action (such as advising the user to immediately go to the hospital, emergently dispatching a first-aid personnel there for giving a first aid or carrying out a surgical operation to the user, etc.) in response thereto, in order not to delay the optimal rescue time.

Therefore, by utilizing the remote health care system, the health care workers can exclude limitations of spaces and distances to observe the user's physiological condition anytime and anywhere and, in the mean time, the user can immediately know his/her own physiological condition and then implement a remedial action by himself/herself. Hence, the users with chronic diseases (such as heart disease, gout, diabetes, liver disease, etc.) or general people still can keep obtaining professional health cares of the health care workers in private residences or some areas lacking medical-resource.

However, there are still some disadvantages existing in the traditional remote health care system, as follows:
(1) Because the symptom diagnosis algorithm is preset in the care server, the user or the health care workers can not select a symptom diagnosis algorithm not preset in the care server for a specific disease through the physiological parameter detection device when the user wants to implement a symptom diagnosis analysis other than those preset in the care server due to his/her symptoms or needs for preventive health care, which not only causes the health care workers unable to provide more complete and comprehensive health care to a variety of physiological conditions of the user, but also causes the user having no way to know his/her own physiological condition more detailed. It eventually results in delaying the optimal rescue time.
(2) Furthermore, because the symptom diagnosis algorithm is preset in the care server, medical experts specializing in various medical fields can not immediately revise or update the symptom diagnosis algorithm. As a result, the care server may continuously use old or existing symptom diagnosis algorithm having errors to analyze the user's physiological parameter data and obtain wrong analysis results, which may eventually cause the health care workers making an erroneous diagnosis to the physiological condition of the user or cause the user making an erroneous judgment about his/her own physiological condition. Thus, the personal safety of the user may be seriously risked, and even irreparable regret may occur.

As a result, it is important for related manufacturers to think how to develop a remote health care system for medical experts specializing in various medical fields to upload various developed symptom diagnosis algorithms to the database of the care server for providing a variety of new and useful symptom diagnosis algorithms to the user, or to revise or update old symptom diagnosis algorithms for the user to select the program files more appropriate to his/her individual diseases, in order to calculate and analyze the user's physiological parameter data by the care server, so that the user and the health care workers can immediately obtain corresponding calculation and analysis results to precisely and objectively understand the user's individual physiological condition.

It is therefore tried by the inventor to develop a remote health care system for enabling a user to select a symptom diagnosis algorithm appropriate to his/her individual disease from a care server for calculating and analyzing the user's physiological parameter data and method thereof to solve the problems existing in the traditional remote health care system, which can not provide a variety of new and useful symptom diagnosis algorithms for being selected by the user according to his/her physical symptoms, as described above.

### BRIEF SUMMARY OF THE INVENTION

A primary object of the present invention is to provide a remote health care system for enabling a user to select a symptom diagnosis algorithm appropriate to his/her individual disease from a care server for calculating and analyzing the user's physiological parameter data, which comprises a care server, at least one first user end (such as a medical expert's computer device) and at least one second user end (such as a user's computer device or communication device), wherein the care server can receive at least one program file (including a symptom diagnosis algorithm) uploaded from each said first user end and allow each said second user end to select each said program file stored in the care server, while the care server can receive at least one set of physiological parameter data (such as electrocardiograms, blood pressure data, blood glucose data, blood oxygen data, etc.) uploaded from each said second user end. Then, the care server can execute a calculation and analysis procedure to each said set of uploaded physiological parameter data by using the symptom diagnosis algorithm in each said selected program file, and transmit a calculation and analysis result (such as a message indicating whether the heartbeat is normal or not) to each said corresponding second user end. As a result, medical experts specializing in various medical fields can upload various developed program files of symptom diagnosis algorithms to a database of the care server for adding, revising or updating each said program file, so that the care server can provide a variety of new and useful program files to the user, and allow the user to adopt the program files more appropriate to his/her individual diseases (such as heart disease, gout, diabetes, liver disease, etc.). Meanwhile, the care server can calculate and analyze each said set of physiological parameter data to precisely and objectively understand the user's individual physiological condition.

A secondary object of the present invention is to provide the remote health care system, wherein each said second user end is provided with a physiological parameter detection device, so that each said set of the user's individual physiological parameter data can be continuously detected and captured by the physiological parameter detection device. Then, each said set of the physiological parameter data can be transmitted to the care server through each said second user end, and the care server can execute an analysis and a calculation thereto, so that the user or the health care workers can immediately obtain corresponding calculation and analysis results to precisely and objectively understand the user's individual physiological condition.

A third object of the present invention is to provide a method applied to the foregoing remote health care system, wherein the medical experts specializing in various medical fields can upload various developed program files of symptom diagnosis algorithms to the care server, so that the user can select the program files more appropriate to his/her individual diseases from the care server. Meanwhile, the physiological parameter data can be uploaded to the care server, and the care server can calculate and analyze the physiological parameter data by using the selected program file, so that the user or the health care workers can immediately obtain corresponding calculation and analysis results from the care server.

### BRIEF DESCRIPTION OF THE SEVERAL VIEWS OF THE DRAWINGS

The structure and the technical means adopted by the present invention to achieve the above and other objects can be best understood by referring to the following detailed description of the preferred embodiments and the accompanying drawings, wherein

FIG. 1 is a block diagram of a remote health care system according to a preferred embodiment of the present invention;

FIG. 2 is a schematic view of an operational window according to the preferred embodiment of the present invention;

FIG. 3 is a schematic view of a calculation and analysis result window according to the preferred embodiment of the present invention;

FIG. 4 is a flowchart of a remote health care method according to a preferred embodiment of the present invention; and

FIG. 5 is a flowchart of another remote health care method according to the preferred embodiment of the present invention.

### DETAILED DESCRIPTION OF THE INVENTION

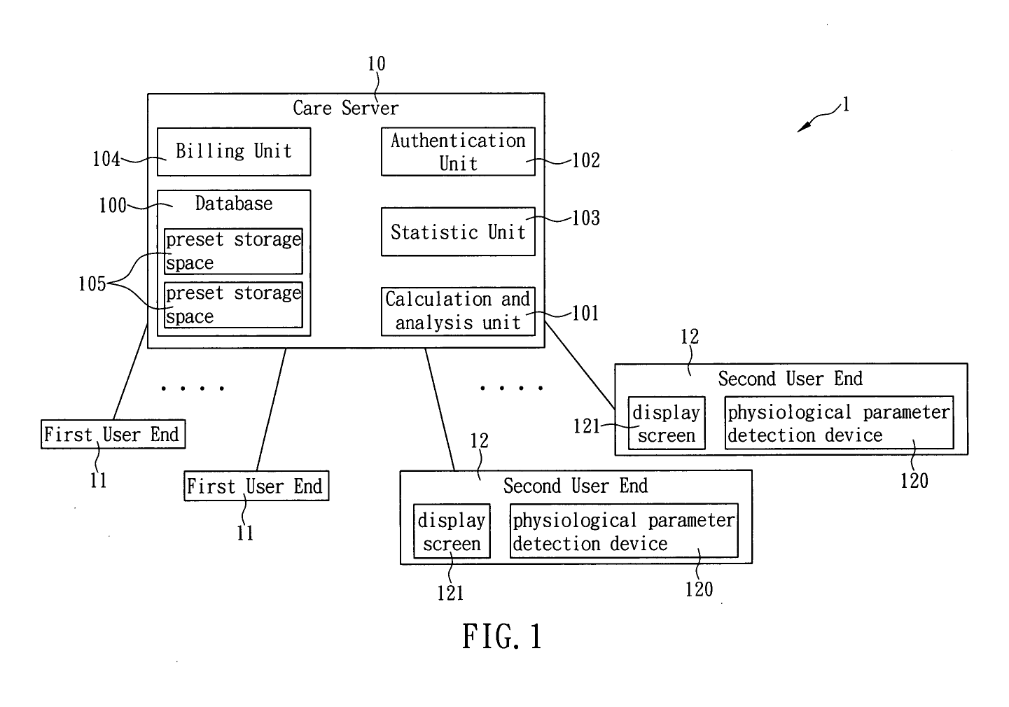

The present invention is a remote health care system for enabling a user to select a symptom diagnosis algorithm appropriate to his/her individual disease from a care server for calculating and analyzing the user's physiological parameter data and a method of the remote health care system. Referring now to FIG. 1, according to a preferred embodiment of the present invention, the remote health care system 1 comprises a care server 10, at least one first user end 11 (such as a medical expert's computer device) and at least one second user end 12 (such as a user's computer device or communication device), wherein the care server 10 is provided with a database 100 which can store a plurality of program files and plural sets of file information including file names, updated dates, related program parameters, used times, operational procedures, application descriptions, etc. Each said first user end 11 can link to the care server 10 via a wired network (such as ADSL, optical fibers, etc.) or a wireless network (such as 802.11, WiMAX, WiFi, 3G etc.). After each said first user end 11 logins the care server 10 and passes an authentication of the care server 10, the first user end 11 can upload at least one program file to the database 100, wherein the program file includes program codes of a symptom diagnosis algorithm (such as an arrhythmia algorithm for calculating and analyzing whether or not the heartbeat frequency is stable within a predetermined duration, a heartbeat algorithm for calculating and analyzing the heartbeat number per minute, etc.), respectively. Meanwhile, each said second user end 12 can also link to the care server 10 via a wired network or a wireless network. After each said second user end 12 logins the care server 10 and passes the authentication of the care server 10, the second user end 12 can select one of said program files from the database 100 through the care server 10 according to the user's desire, and upload at least one set of physiological parameter data (such as electrocardiograms, blood pressure data, blood glucose data, blood oxygen data, etc.) to the care server 10. Thus, a calculation and analysis unit 101 of the care server 10 can execute a calculation and analysis procedure to each said set of uploaded physiological parameter data according to the algorithms in the selected program file. Then, the care server 10 will transmit a calculation and analysis result (such as a message indicating whether the heartbeat frequency is normal or not) with an image format or a text format to the corresponding second user end 12.

Therefore, medical experts specializing in various medical fields can upload various developed program files of symptom diagnosis algorithms to the database 100 of the care server 10 through the remote health care system 1 of the present invention for adding, revising or updating the program files, so that the care server 10 can provide a variety of new and useful program files to the user, and allow the user to select and adopt the program files more appropriate to his/her individual diseases (such as heart disease, gout, diabetes, liver disease, etc.). Meanwhile, the care server 10 can calculate and analyze each said set of the physiological parameter data uploaded by the user, so that the user or the health care workers can immediately obtain corresponding calculation and analysis results from the care server 10 to precisely and objectively understand the user's individual physiological condition. As a result, the health care workers can immediately implement an appropriate therapy and action (such as advising the user to immediately go to the hospital, emergently dispatching a first-aid personnel there for giving a first aid or carrying out a surgical operation to the user, etc.) in response thereto, and the user can implement corresponding remedial actions (such as sports, food therapies, drug administration, hospital treatment, surgical operations, etc.) by himself/herself, so that the user or the health care workers can efficiently hold the optimal time for treatment and rescue.

In the embodiment, referring to FIG. 1, each said second user end 12 is provided with a physiological parameter detection device 120 for detecting and capturing the user's individual physiological parameter data. The physiological parameter detection device 120 can be designed as a portable device (such as integrated in a mobile phone, a PDA or other portable electronic devices), so that the user can carry the physiological parameter detection device 120 to continuously detect and capture the individual physiological parameter data in a real-time manner. Then, each said second user end 12 can be used to transmit the physiological parameter data to the care server 10, and the care server 10 can execute an analysis and a calculation, so that the user and the health care workers can immediately obtain corresponding calculation and analysis results from the care server 10 to precisely and objectively understand the user's individual physiological condition.

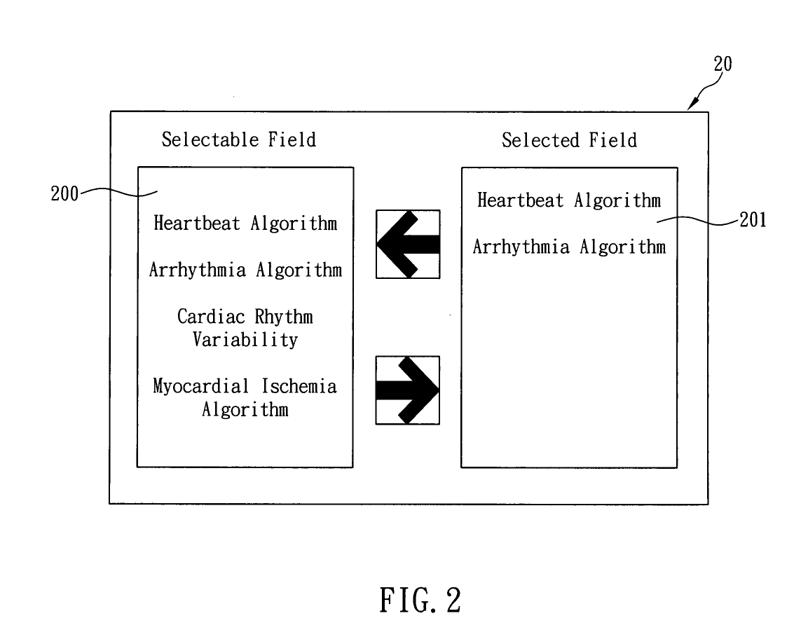

Moreover, referring now to FIGS. 1 and 2, in the embodiment, each said second user end 12 is provided with a display screen 121. When the user wants to select the program file through each said second user end 12, each said second user end 12 can read the program file in the database 100, and list and show file information of the program files (such as sorting by prefixes of each of the symptoms, affected areas corresponding to each of the symptoms, historical utilization rates of each of the program files, used evaluations of each of the program files, etc.) in series on a selectable field 200 in an operational window 20 of the display screen 121 of each said second user end 12, so that the user can select the file information. In addition, the operation window 20 further comprises a selected field 201. Thus, after the user selects one of the program files, the file information of the selected program file will be listed and shown on the selected field 201 in series for the user's confirmation. As a result, the user not only can use the second user end 12 to archive and list all of the usable program files in the database 100 for conveniently adopting the program files via the simple operational window 20 according to the user's desire, but also can prevent the problem of incorrect or repeated selections due to being unable to confirm which program files have already been selected.

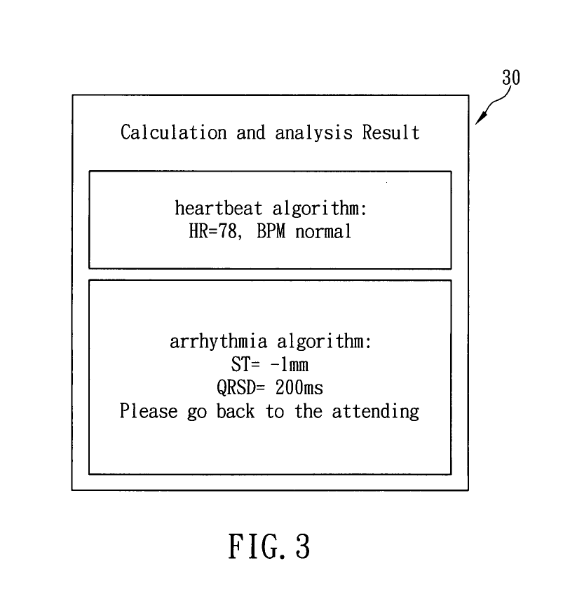

In the embodiment, referring now to FIGS. 1 and 3, after the calculation and analysis unit 101 of the care server 10 executes the calculation and analysis procedure to the uploaded physiological parameter data according to the algorithm in the selected program file and finishes the calculation and analysis procedure, the care server 10 will transmit the calculation and analysis result to the second user end 12. Then, the second user end 12 can show the calculation and analysis result on a calculation and analysis result window 30 of the display screen 121. In such a way, the user can immediately obtain the individual physiological condition from the calculation and analysis result window 30, and implement corresponding remedial actions by himself/herself, so that there will be better guarantee for the health condition and the personal safety of the user.

Furthermore, in the embodiment, the database 100 further comprises at least one preset storage space 105 for storing the calculation and analysis results of each said second user end 12, respectively. Thus, after the calculation and analysis unit 101 of the care server 10 finishes the calculation and analysis procedure, the calculation and analysis unit 101 can store the calculation and analysis results in the preset storage space 105 of the database 100 corresponding to each said second user end 12. After this, the user can access and archive the calculation and analysis results and provide the results to a medical personnel for making a professional diagnosis according to the results, so that the medical personnel can efficiently execute diagnosis operations to confirm the health condition of the user.

Referring still to FIG. 1, in the embodiment, the care server 10 further comprises an authentication unit 102 to execute an authentication procedure to each said algorithm when each said first user end 11 uploads each said program file to the database 100. The authentication unit 102 will delete some of the program files which fail to pass the authentication procedure, wherein the authentication procedure at least comprises one of the three authentication methods, as follows:
(1) Verifying whether the processing efficiency of each said program file fits a predetermined processing efficiency or not;
(2) Verifying whether each said program file passes an international standard certification or not; and
(3) Verifying whether each said program file has no virus code endangering the care server 10 or not.

Therefore, the authentication procedure of the authentication unit 102 to each said program file can effectively ensure the operational quality and efficiency of the remote health care system 1, so as to guarantee the rights and interests of each of the users using the remote health care system 1.

Besides, in the embodiment, referring back to FIGS. 1 and 2, the care server 10 further comprises a statistic unit 103 to calculate and analyze the number of the program files used by each said second user end 12, respectively, and to convert number of times of use into file information of the corresponding program files. Thus, when the user selects each said program file, the user can view the number of times of use (i.e. utilization rate) of the program file in the past through the display screen 121 of each said second user end 12, so that the number of times of use can be used as a reference for the user to use the program file.

Referring back to FIG. 1, in the embodiment, the care server 10 further comprises a billing unit 104. When the user uses each said program file to detect the individual physiological condition through each said second user end 12 (or when the user uses each said second user end 12 to link to the care server 10), the billing unit 104 can execute a billing procedure, so that related manufacturers providing the remote health care system 1 or medical experts providing the program files can obtain corresponding remuneration according to a calculated and analyzed billing result.

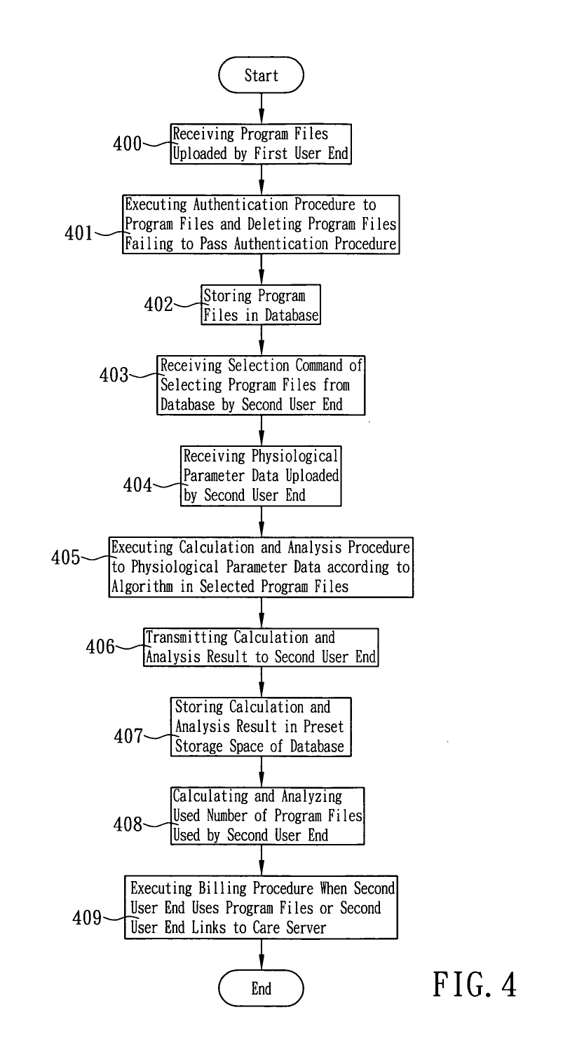

In addition, in the embodiment, referring now to FIGS. 1 and 4, the method is applied to the remote health care system 1, and controls the care server 10 to execute the steps, as follows:

In a step 400, receiving each said program file uploaded by each said first user end 11;

In a step 401, executing an authentication procedure to each said program file by an authentication unit 102, and deleting some of the program files which fail to pass the authentication procedure;

In a step 402, storing each said authenticated program file in the database 100;

In a step 403, receiving a selection command of selecting each said program file from the database 100 by each said second user end 12;

In a step 404, receiving at least one set of physiological parameter data uploaded by each said second user end 12;

In a step 405, executing a calculation and analysis procedure to each said set of physiological parameter data according to at least one algorithm in each said selected program file through the calculation and analysis unit 101, so as to calculate and analyze a physiological condition of the user;

In a step 406, transmitting a calculation and analysis result with an image format or a text format to the corresponding second user end 12;

In a step 407, storing the calculation and analysis result with the image format or the text format in a preset storage space 105 of the database 100 corresponding to the second user end 12;

In a step 408, calculating and analyzing a number of each said program file used by each said second user end 12 through a statistic unit 103, respectively; and

In a step 409, executing a billing procedure by a billing unit 104 when each said second user end 12 uses each said program file or when each said second user end 12 links to the care server 10.

Referring still to FIGS. 1 and 4, in the embodiment, in order to shorten and simplify the procedures of the foregoing embodiments to enhance the execution efficiency of the care server 10, the steps 400 through 402 (i.e. receiving the program file uploaded by each said first user end 11, executing the authentication procedure, and storing the program file in the database 100) as shown in FIG. 4 may be omitted in a case that the program files in the database 100 are adequate and all the program files are finalized, or in a case that no said program file needs to be updated or revised within a short time. In such a way, the care server 10 can simply process the selection command from each said second user end 12 and each said set of physiological parameter data, and execute the calculation and analysis procedure without receiving or processing each said program file from each said first user end 11, so that the operational loading of the care server 10 can be efficiently lowered.

Furthermore, in the embodiment, referring now to FIGS. 1, 2 and 5, the method can further allow each said second user end 12 to execute the steps, as follows:

In a step 500, reading the program files in the database 100, and listing and showing file information of the program files (such as sorting by prefixes of each of the symptoms, affected areas corresponding to each of the symptoms, historical utilization rates of each of the program files, used evaluations of each of the program files, etc.) in series on a selectable field 200 in an operational window 20 of the display screen 121 of each said second user end 12, so that the user can select the file information; and

In a step 501, listing and showing the file information of each said selected program file on the selected field 201 of the operational window 20 in series after the user selects the program file for the user's confirmation.

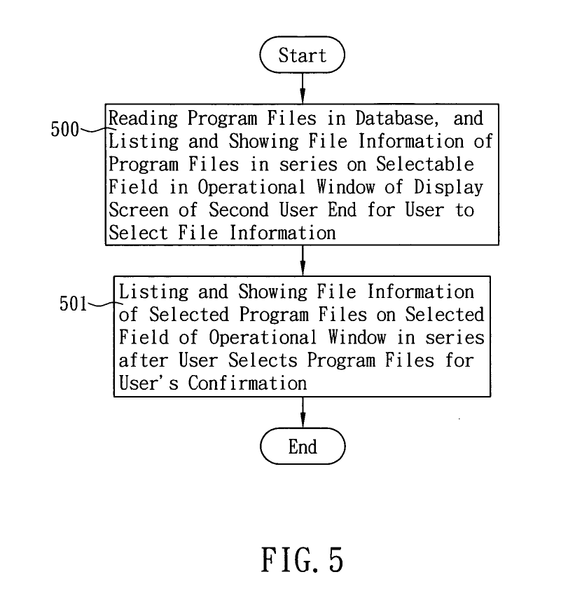

Moreover, referring still to FIGS. 1 and 5, in the embodiment, if each said second user end 12 is provided with fixed selection buttons for the user to select the program files, the steps 500 and 501 as shown in FIG. 5 can be omitted because the user can directly know what is the selected program file from the selection buttons selected by the user. As a result, the manufacturers can reduce the material cost of fabricating the display screen 121, while the loading of data transmission between each said second user end 12 and the care server 10 can be lowered.

As described above, as shown in FIG. 1, the present invention can mainly allow medical experts specializing in various medical fields to upload various developed program files of symptom diagnosis algorithms to the database 100 of the care server 10, so that the care server 10 can provide a variety of new and useful program files to the user, and allow the user to select the program files more appropriate to his/her individual diseases from the care server 10 and, in the meantime, to upload the physiological parameter data to the care server 10, so that the care server 10 can execute a calculation and analysis procedure thereto. Thus, the user or the health care workers can immediately obtain corresponding calculation and analysis results to precisely and objectively understand the user's individual physiological condition.

The present invention has been described with a preferred embodiment thereof and it is understood that many changes and modifications to the described embodiment can be carried out without departing from the scope and the spirit of the invention that is intended to be limited only by the appended claims.

What is claimed is:

1. A remote health care method for enabling a user to select a symptom diagnosis algorithm appropriate to his/her individual disease, which is applied to a remote health care system comprising a care server, at least one first user end and at least one second user end, wherein the care server is provided with a database which stores a plurality of program files and plural sets of file information, each said first user end and second user end link to the care server via a network, respectively, each said first user end uploads at least one program file to be stored in the database, and each said program file includes program codes of a symptom diagnosis algorithm, the method controlling the care server to execute steps comprising:
receiving a selection command of selecting each said program file from the database by each said second user end;
receiving at least one set of physiological parameter data uploaded by each said second user end;
executing a calculation and analysis procedure to each said set of physiological parameter data according to the algorithm in each said selected program file through a calculation and analysis unit; and
transmitting a calculation and analysis result obtained by said calculation and analysis unit to the corresponding second user end.

2. The remote health care method according to claim 1, wherein the method further controls the care server to execute steps of:
receiving each said program file uploaded by each said first user end; and
storing each said program file in the database.

3. The remote health care method according to claim 1, wherein the method further allows each said second user end to execute a step of:
reading the program files in the database, and listing and showing file information of each said program file in series on a selectable field in an operational window of a display screen of each said second user end, so that the user is allowed to select the file information.

4. The remote health care method according to claim 2, wherein the method further allows each said second user end to execute a step of:
reading the program files in the database, and listing and showing file information of each said program file in series on a selectable field in an operational window of a display screen of each said second user end, so that the user is allowed to select the file information.

5. The remote health care method according to claim 3, wherein the operation window further comprises a selected field, and the method further allows each said second user end to execute a step of:
listing and showing the file information of each said selected program file on the selected field in series after the user selects each said program file for the user's confirmation.

6. The remote health care method according to claim 4, wherein the operation window further comprises a selected field, and the method further allows each said second user end to execute a step of:
listing and showing the file information of each said selected program file on the selected field in series after the user selects each said program file for the user's confirmation.

7. The remote health care method according to claim 5, wherein after receiving each said program file uploaded by each said first user end, the method further controls the care server to execute a step of:
executing an authentication procedure to each said program file by an authentication unit, and deleting some of the program files which fail to pass the authentication procedure.

8. The remote health care method according to claim 6, wherein after receiving each said program file uploaded by each said first user end, the method further controls the care server to execute a step of:
executing an authentication procedure to each said program file by an authentication unit, and deleting some of the program files which fail to pass the authentication procedure.

9. A remote health care system for enabling a user to select a symptom diagnosis algorithm appropriate to his/her individual disease, comprising:
a care server provided with a database which stores a plurality of program files and a plurality of file information;
at least one first user end linking to the care server via a network, wherein each said first user end uploads at least one program file to the database, while each said program file includes program codes of a symptom diagnosis algorithm; and
at least one second user end linking to the care server via a network, wherein each said second user end selects each said program file from the database through the care server and uploads at least one set of physiological parameter data to the care server, so that a calculation and analysis unit of the care server executes a calculation and analysis procedure to each said set of uploaded physiological parameter data according to the algorithms in each said selected program file, and transmit a calculation and analysis result to the corresponding second user end.

10. The remote health care system according to claim 9, wherein each said second user end is provided with a physiological parameter detection device for detecting and capturing the physiological parameter data.

11. The remote health care system according to claim 10, wherein each said second user end is provided with a display screen for showing file information of the program files read by the second user end from the database, and listing and showing file information of the program file in series on a selectable field in an operational window of the display screen for the user to select the file information, or showing a calculation and analysis result transmitted from the care server.

12. The remote health care system according to claim 11, wherein the operation window further comprises a selected field for showing the file information of each said selected program file, and the file information is listed and shown on the selected field in series for the user's confirmation.

13. The remote health care system according to claim 12, wherein the care server further comprises an authentication unit to execute an authentication procedure to each said program file when each said first user end uploads each said program file to the database, and delete some of the program files which fail to pass the authentication procedure.

14. The remote health care system according to claim 12, wherein the care server further comprises a statistic unit to calculate and analyze a number of each said program file used by each said second user end.

15. The remote health care system according to claim 12, wherein the care server further comprises a billing unit for executing a billing procedure when each said second user end uses each said program file or when each said second user end links to the care server.

16. The remote health care system according to claim 12, wherein the database further comprises at least one preset storage space for storing the calculation and analysis result of each said second user end, respectively.
